(19) United States
(12) Patent Application Publication
(10) Pub. No.: US 2025/0090827 A1
(43) Pub. Date: Mar. 20, 2025

(54) RELEASABLE COUPLING FOR A MEDICAL DEVICE

(71) Applicants: Boston Scientific Scimed, Inc., Maple Grove, MN (US); Boston Scientific Medical Device Limited, Galway (IE)

(72) Inventors: Nachiket GOLE, Pune (IN); Pooja Bhuvanesh KULKARNI, Pune (IN); Barry WEITZNER, Acton, MA (US); Deepak Kumar SHARMA, Muzaffarnagar (IN); Aditya DHANOTIYA, Indore (IN); Subodh MOREY, Ponda (IN)

(73) Assignees: Boston Scientific Scimed, Inc., Maple Grove, MN (US); Boston Scientific Medical Device Limited, Galway (IE)

(21) Appl. No.: 18/887,309

(22) Filed: Sep. 17, 2024

Related U.S. Application Data
(60) Provisional application No. 63/583,660, filed on Sep. 19, 2023.

(51) Int. Cl. A61M 39/10

(52) U.S. Cl. CPC A61M 39/10

(57) ABSTRACT

A medical device may comprise a proximal component. The proximal component may comprise a handle with a coupling portion. The coupling portion is configured to releasably couple to a distal joint of a distal component of the medical device. A proximal wire may extend distally from the handle. The distal end of the proximal wire may include a connector that may be configured to releasably connect to a distal wire of the distal component of the medical device.

### CROSS-REFERENCE TO RELATED APPLICATIONS

This application claims the benefit of priority to U.S. Provisional Application No. 63/583,660, filed on Sep. 19, 2023, which is incorporated by reference herein in its entirety.

### TECHNICAL FIELD

The disclosure relates generally to a coupling for medical devices. More specifically, aspects of the disclosure pertain to a releasable coupling for attaching a proximal portion of a medical device to a distal portion of the medical device.

### BACKGROUND

Single-use medical devices can result in a large amount of waste, creating a burden on the environment. For example, many endoscopic devices, including for example hemostasis clips and biopsy forceps, are currently single-use devices. Such devices often include a handle, a shaft, and a distal tip that includes an end effector. The distal tip and the shaft are inserted in the body to perform a procedure at a tissue site (e.g., obtain a tissue sample), while the handle remains outside of the body. Disposal of the entire device creates large amounts of waste and environmental burden. Therefore, there is a need for an apparatus that results in less waste and environmental burden. The apparatus and methods described herein may alleviate this deficiency and one or more other deficiencies in the art. However, the attached claims specify the scope of this disclosure rather than the ability to solve any specific problem.

### SUMMARY

This disclosure includes devices and methods that allow for releasably coupling a proximal component of a medical device (including a handle) to a distal component of the medical device (including portions that are inserted in the body during use). Such devices and methods permit, for example, re-use of the proximal component with other distal components, resulting in less waste. Each of the aspects disclosed herein may include one or more of the features described in connection with any of the other disclosed aspects. The examples described herein may have any of these features in any combination.

A medical device may comprise a proximal component. The proximal component may include a handle with a coupling portion. The coupling portion is configured to releasably couple to a distal joint of a distal component of the medical device. A proximal wire may extend distally from the handle. The distal end of the proximal wire may include a connector that may be configured to releasably connect to a distal wire of the distal component of the medical device.

The handle may include a handle body and an actuator that is movable relative to the handle body. The proximal end of the proximal wire may be fixed to an actuator.

The proximal component of the medical device may include a connector that includes a hook. The hook may have a first end fixed to a face of a body of the connector. The hook may have a free end and a curved portion of the hook may be between the first end and the free end. A space may be defined between the free end and a distal face of the body.

The proximal component of the connector may include a loop defined by a proximal body, a pin and two arms connecting the proximal body to the pin. The two arms may be spaced apart. The arms may extend longitudinally from a distal face of the body, and the pin may span a first space between the two arms.

The first space may be proximal to the pin. The connector may define a second space distal to the pin and between the pin and a distal body of the connector. The pin may be between a distal face of the proximal body and a proximal face of the distal body. The proximal component may further include a proximal shaft portion over the proximal wire. The proximal wire may translate relative to the proximal shaft portion.

The coupling portion may include a coupling portion at a distal end of the handle. The coupling portion may include a proximal joint that slides over the proximal shaft portion and the proximal wire. The proximal joint may include an outer wall and an inner wall defining a sleeve. The outer wall may connect to the sleeve at a proximal end of the proximal joint, and a space may be between the outer wall and the sleeve. The sleeve may surround and contact the connector and the proximal joint may rotate relative to the proximal shaft portion.

The proximal joint may include a flange extending radially inward from the inner wall and defining a distally facing wall. The flange may define a proximal opening of the proximal joint. The proximal opening may receive the proximal wire.

The medical device may include a distal component including an end effector. A distal wire may extend proximally from the end effector. A proximal end of the distal wire may include a connector that may be configured to releasably connect to a proximal wire of the proximal component of the medical device. A distal shaft portion may be over the distal wire, and the distal wire may translate relative to the distal shaft portion.

The distal joint may be configured to releasably couple to a proximal joint of a proximal component of the medical device. The distal joint may translate over the distal shaft portion. The distal component of the medical device may include the connector including one of a hook and a loop. The distal component of the medical device may include an outer wall and an inner wall defining a sleeve. The outer wall may connect to the sleeve at a distal end of the distal joint. A space may be between the outer wall and the sleeve, and the distal joint may define a distal opening that receives the distal wire.

The medical device may include a proximal handle and a distal end effector. The proximal wire may extend distally from the handle and a distal end of the proximal wire may include a first connector. A distal wire may extend proximally from the distal end effector and a proximal end of the distal wire may include a second connector to releasably connect to the first connector.

A proximal coupling portion may be over the proximal wire. A distal joint may translate over the distal wire and may be configured to releasably couple to the proximal coupling. The first connector may include a pin and a hook and the second connector may include the other of the pin and the hook.

It may be understood that both the foregoing general description and the following detailed description are exemplary and explanatory only and are not restrictive of the invention, as claimed. As used herein, the terms “comprises,” “comprising,” “has,” “having,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/−10% of a stated value.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1 illustrates a perspective view of an exemplary medical device, including an enlarged view of a distal portion of the medical device, according to an embodiment;

FIG. 2A-2C illustrate perspective views of various aspects of the distal portion of the medical device of FIG. 1;

FIG. 3 illustrates a partially cutaway view of a proximal portion of the medical device of FIG. 1;

FIG. 4 illustrates a perspective view a connected assembly of the medical device of FIG. 1, in a final connected form;

FIG. 5 illustrates a perspective, cross-sectional view of the connected assembly of FIG. 4;

FIGS. 6A-6C illustrate perspective, side, and top views respectively of first and second connectors of the connected assembly of FIG. 5;

FIG. 7 illustrates a perspective view of the first connector;

FIG. 8 illustrates a perspective view of the second connector;

FIG. 9A-9E illustrate an exemplary method of connecting the first connector to the second connector, during different stages of assembly;

FIG. 10A illustrates a side view of another exemplary coupling of a medical device, according to an embodiment; and

FIG. 10B illustrates a perspective view of the coupling of FIG. 10A, with a distal joint exposing portions of the coupling.

### DETAILED DESCRIPTION

Reference is now made in detail to examples of this disclosure, aspects of which are illustrated in the accompanying drawings. Wherever possible, the same reference numbers will be used throughout the drawings to refer to the same or like parts.

Embodiments of this disclosure include a medical device that has a proximal component that releasably couples to a distal component. The proximal component may include a handle, a proximal portion of a shaft, and a proximal portion of a wire. The distal component may include a distal portion of the shaft, a distal portion of the wire, and an end effector. Prior to use of the medical device, the proximal portion of the wire may releasably attach to the distal portion of the wire, and the proximal portion of the shaft may releasably couple to the distal portion of the shaft, so that actuation of the handle may actuate the end effector. After use, the proximal portion of the wire may be de-coupled from the distal portion of the wire, and the proximal portion of the shaft may be de-coupled from the distal portion of the shaft. In this way, the distal component, which contacts the patient during use, may be disposed of, and the proximal component may be cleaned as needed and re-used with a second distal component in a subsequent procedure.

Such embodiments of a medical device may result in reduced waste and reduced overall cost of the medical device (less handles needed, less packaging, etc.). Such embodiments also may result in a universal proximal component (including a handle) that may be used with numerous different types of distal components (including different types of end effectors).

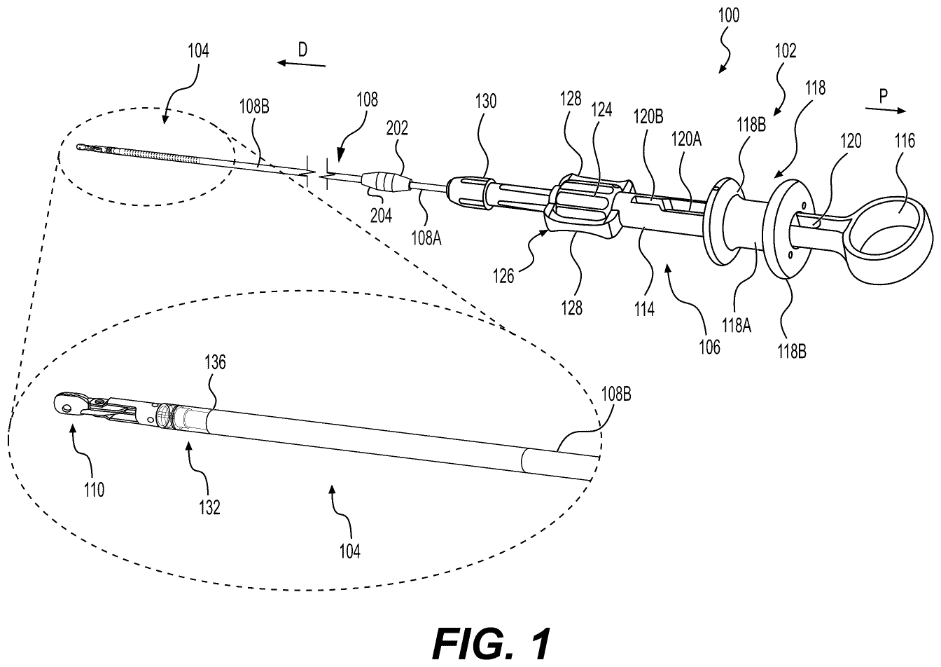

FIG. 1 illustrates a medical device 100, including a proximal portion 102 and a distal portion 104. FIG. 1 includes an enlarged view of distal portion 104. As shown in FIG. 1, proximal portion 102 includes a handle 106. Medical device 100 includes a shaft 108, for example, extending from a distal end of handle 106. Shaft 108 includes a proximal shaft portion 108A and a distal shaft portion 108B that releasably couple to each other, as described below. Shaft 108 may include or otherwise radially surround a wire 138, for example, a pull wire or an actuation wire. For example, wire 138 may extend from handle 106 through shaft 108, and wire 138 may be movable (e.g., within shaft 108) via manipulation of one or more portions of handle 106 to control one or more aspects of distal portion 104 of medical device 100, for example, including an end effector 110. In embodiments, wire 138 includes a proximal wire portion 138A and a distal wire portion 138B that releasably couple to each other, as described below.

Medical device 100 includes end effector 110 at a distal end of shaft 108, for example, at distal portion 104. Medical device 100 may be delivered to the treatment site via an insertion device (e.g., through a working channel of an endoscope, ureteroscope, catheter, etc.).

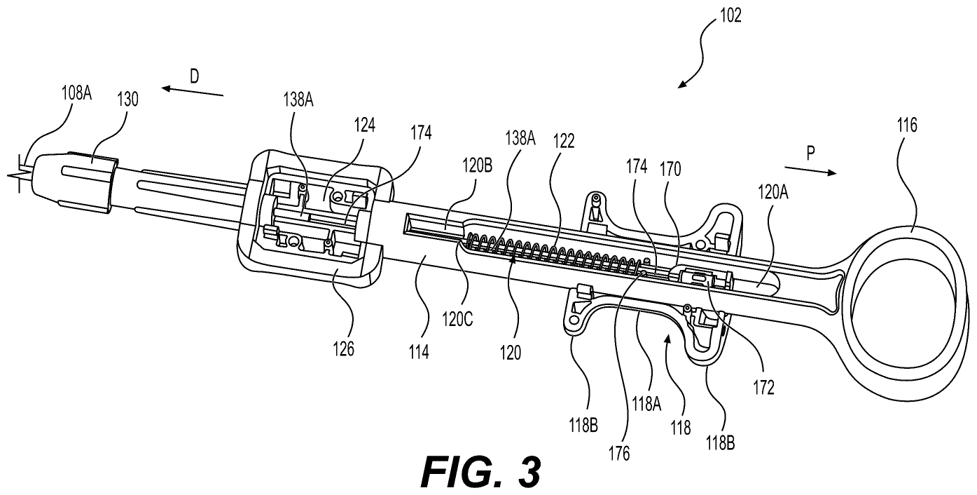

Handle 106 includes a main body 114, for example, including a ring 116 (e.g., a thumb ring), for example, at a proximal end of main body 114. Handle 106 also includes a first movable member or spool 118. Spool 118 may be an actuator and may include an indented portion 118A (e.g., with a relatively smaller lateral cross-section) and one or more (e.g., two) ridged or extended portions 118B (e.g., with a relatively larger lateral cross-section). In these aspects, indented portion 118A may receive one or more of the user's fingers, such that movement of the user's finger(s) controls the movement of spool 118. Main body 114 may include a slot 120, for example, extending longitudinally through a portion of main body 114, for example, from a position spaced distally from ring 116. A portion of spool 118 may extend into a portion of slot 120, such that spool 118 is movable (e.g., longitudinally movable distally and/or proximally) along slot 120. In these aspects, slot 120 may define a range of movement for spool 118. As discussed in detail below, spool 118 may be movable within slot 120, for example, proximally and/or distally, to control one or more aspects of end effector 110. Furthermore, slot 120 may include a wide portion 120A and a tapered or narrow portion 120B. As discussed below, wide portion 120A may be proximal of narrow portion 120B, and the transition between wide portion 120A and narrow portion 120B may support or otherwise provide a stop surface for a biasing element 122 (FIG. 3, discussed below).

Additionally, handle 106 includes a second movable member or knob 124. For example, main body 114 may include a cage 126, for example, formed by two arms 128 that partially surround knob 124. Knob 124 and cage 126 may be distal of slot 120 on main body 114. As discussed below, knob 124 may be an actuator and may be rotatable, for example, clockwise and/or counterclockwise, to control one or more aspects of end effector 110. For example, knob 124 may be rotatable about a longitudinal axis (e.g., a central longitudinal axis) of medical device 100.

Handle 106 may also include an end cap 130, for example, at a distal end of handle 106. End cap 130 may surround a proximal end of proximal shaft portion 108A. End cap 130 may help to couple proximal shaft portion 108A to handle 106. In some aspects, end cap 130 may form a strain relief portion of medical device 100. End cap 130 may couple to a distal end of main body 114 in a manner that permits relative rotation therebetween.

Medical device 100 may include end effector 110 at distal portion 104. As shown, end effector 110 may be a forceps, for example, including two jaws. Nevertheless, this disclosure is not so limited, and end effector 110 may be any type of end effector, instrument, tool, or other device (e.g., grasper, snare, basket, hemostatic clip, stapler, ablation device, tome, suturing device, needle, knife, etc.).

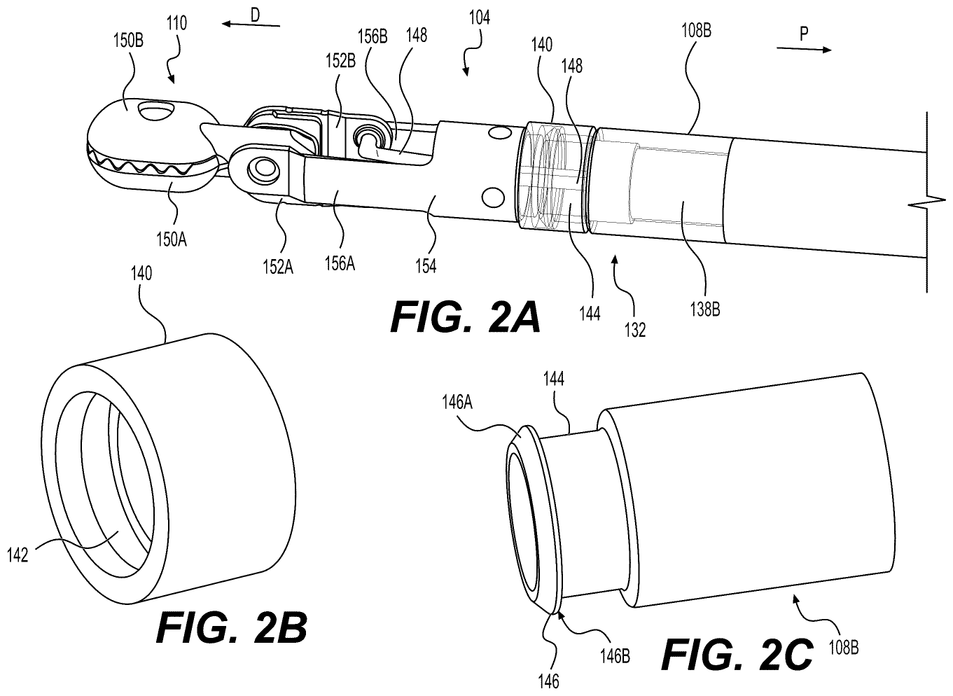

End effector 110 may be coupled to shaft 108 at the distal end of distal shaft portion 108B, for example, at a coupling portion 132. Coupling portion 132 may include a bushing or bush 140, which may be generally cylindrical and may also include an undercut 142 (FIG. 2B). Undercut 142 may be formed by a reduced radial thickness along an inner portion of bush 140, for example, extending circularly along the inner portion of bush 140. Additionally, a proximal portion of end effector 110 (specifically a clevis, as mentioned below) may be coupled to bush 140, for example, via a weld.

Distal shaft portion 108B may include a distal extension 144, for example, including or being coupled to a flange portion 146, as shown in FIG. 2C. As shown, distal extension 144 may be smaller than flange portion 146. For example, both distal extension 144 and flange portion 146 may be generally circular in a lateral cross-section, and distal extension 144 may include a smaller circumference than flange portion 146. Flange portion 146 may be positioned within undercut 142, for example, to rotatably couple shaft 108 to bush 140. For example, a distal portion 146A of flange portion 146 may include a beveled, slanted, or tapered surface, which may assist flange portion 146 to be positioned within undercut 142. Flange portion 146 includes a flat proximal portion 146B, and flange portion 146 may be positioned within undercut 142, to couple bush 140 and distal extension 144 (and thus bush 140 and shaft 108). In these aspects, distal shaft portion 108B may be rotatably coupled to bush 140, while distal shaft portion 108B also fixed longitudinally. That is, bush 140 can rotate relative to distal shaft portion 108B, but bush 140 does not move longitudinally relative to distal shaft portion 108B.

Additionally, distal wire portion 138B may be coupled to end effector 110, for example, via one or more end effector wires 148. The one or more end effector wires 148 (in this case, two wires 148, one wire 148 corresponding to each jaw 150A, 150B) may extend within bush 140. In these aspects, rotation of wire 138 (including distal wire portion 138B) about its axis rotates end effector 110, and longitudinal movement of wire 138 (including distal wire portion 138B) controls or manipulates one or more aspects of end effector 110. For example, as discussed below, wire 138 and end effector wire(s) 148 may rotate end effector 110. Additionally, end effector 110 may be coupled to bush 140, which is rotatably coupled to distal shaft portion 108B.

As shown in FIG. 2A, end effector 110 may include or otherwise be coupled to one or more end effector controls, for example, end effector wires 148. The one or more end effector wires 148 may be operably coupled to distal wire portion 138B. For example, proximal end(s) of each end effector wire(s) 148 may be fixedly coupled to a distal end of distal wire portion 138B (e.g., either directly or indirectly). In these aspects, movement of wire 138 (including distal wire portion 138B) may control the movement of the one or more end effector wires 148, for example, to open and/or close or otherwise manipulate end effector 110. As shown, end effector 110 may be forceps, and may include a pair of jaws 150A, 150B. Movement of the one or more end effector wires 148 may open or close one or more of jaws 150A, 150B, for example, by pivoting jaws 150A, 150B such that distal ends of jaws 150A, 150B move away from each other (e.g., to open) or toward each other (e.g., to close). In some aspects, medical device 100 may include one end effector wire 148, for example, coupled to one of jaws 150A or 150B, such that movement of distal wire portion 138B moves the one end effector wire 148 to move one of jaws 150A or 150B away from the other of the jaws 150A or 150B. In other aspects, medical device 100 may include two end effector wires 148. In this example, one end effector wire 148 may be coupled to jaw 150A. Another end effector wire 148 may be coupled to jaw 150B, such that movement of distal wire portion 138B moves each of the two end effector wires 148 to move both of jaws 150A and 150B away from each other.

In some aspects, each of jaws 150A, 150B may include a proximal leg 152A, 152B. End effector 110 may include a proximal support 154 (also called a clevis), and proximal support 154 may include two distally extending posts 156A, 156B. Proximal legs 152A, 152B may be pivotably connected to respective posts 156A, 156B. In some aspects, one or more portions of end effector 110 (e.g., end effector wires 148) may be biased toward a configuration, for example, toward the closed configuration shown in FIG. 2A. Furthermore, a proximal end of end effector 110 (e.g., a proximal end of proximal support 154) may be coupled to bush 140, for example, welded or otherwise securely coupled (e.g., via a snap fit, a press fit, an adhesive, etc.).

A proximal end of proximal wire portion 138A connects with spool 118, such that movement of spool 118 controls an extension and/or retraction of wire 138 within shaft 108.

FIG. 3 is a partially cutaway view of proximal portion 102 of medical device 100 of FIG. 1, including handle 106. Handle 106 includes spool 118 that is movable relative to main body 114, for example, within a portion of slot 120. Additionally, handle 106 includes a spring or biasing element 122, for example, at least partially positioned within slot 120, using the transition from wide portion 120A to narrow portion 120B as a distal stop surface 120C for biasing element 122. Spool 118 may include an internal projection 170, for example, that extends within a portion of slot 120. Internal projection 170 or another portion of spool 118 may be coupled to a proximal end of proximal wire portion 138A, for example, via a crimp 172 (e.g., a cylindrical crimp) around an end of proximal wire portion 138A. A proximal end of biasing element 122 may be coupled to a distal end of projection 170. Alternatively, biasing element 122 may not be coupled to the distal end of projection 170, but may abut or otherwise interact with the distal end of the projection 170 as spool 118 is advanced distally within slot 120. Biasing element 122 may bias spool 118 proximally. For example, biasing element 122 may compress as spool 118 is moved distally, and biasing element 122 may urge spool 118 proximally (e.g., to the position shown in FIG. 3) once the distal force on spool 118 is removed.

Moreover, as shown in FIG. 3, a proximal wire portion 138A may be coupled to or otherwise interact with knob 124. For example, knob 124 may be formed of two halves that are coupled together to surround part of proximal wire portion 138A. Additionally, a crimp or tube, for example, a hypotube 174, may be attached or otherwise positioned around part of proximal wire portion 138A. Hypotube 174 may include one or more flat outer surfaces, for example, including a square cross-section with four flat outer surfaces. Knob 124 may surround proximal wire portion 138A and hypotube 174, such that knob 124 engages with hypotube 174 to help rotate hypotube 174 and proximal wire portion 138A as knob 124 rotates.

In these aspects, rotation of knob 124 (e.g., clockwise or counterclockwise) may also rotate proximal wire portion 138A, and thus also end effector 110 (e.g., clockwise or counterclockwise about central a longitudinal axis of wire 138 and/or a central longitudinal axis of shaft 108), for example, via the connection via end effector wire(s) 148. For example, the user may rotate knob 124 (e.g., relative to cage 126 about a central longitudinal axis of knob 124) to rotate proximal wire portion 138A (via hypotube 174) and end effector 110 to orient or otherwise position end effector 110 at the treatment site. In these aspects, the user may rotate knob 124 clockwise, which also rotates wire 138 and end effector 110 clockwise. Similarly, the user may rotate knob 124 counterclockwise, which also rotates wire 138 and end effector 110 counterclockwise. In these aspects, the rotation of knob 124, and thus of wire 138 and end effector 110, is independent of any rotation of shaft 108.

In addition, movement of spool 118 may control the actuation of end effector 110. For example, longitudinal movement of spool 118 in a first direction (e.g., in the distal direction) may open jaws 150A, 150B of end effector 110.

As described above, prior to use of medical device 100, proximal wire portion 138A may releasably attach to distal wire portion 138B, and proximal shaft portion 108A may releasably couple to distal shaft portion 108B, so that actuation of handle 106 may actuate end effector 110. After use, proximal wire portion 138A may be de-coupled from distal wire portion 138B, and proximal shaft portion 108A may be de-coupled from distal shaft portion 108B. In this way, the distal components, including distal wire portion 138B, distal shaft portion 108B, and end effector 110, which may contact the patient during use, may be disposed of. Proximal components, including proximal wire portion 138A, proximal shaft portion 108A, and handle 106, may be cleaned as needed and re-used with other distal components in a subsequent procedure.

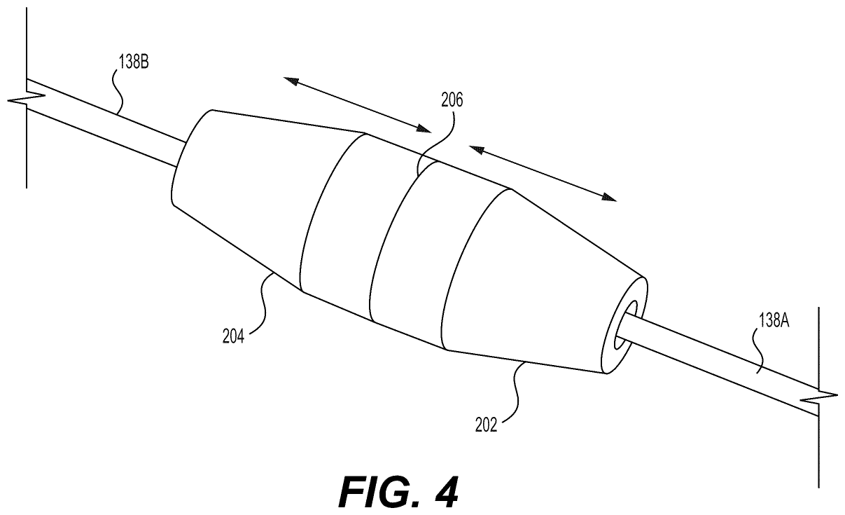

FIGS. 4 to 9E show a connection assembly 600, and a method for releasably attaching or coupling the proximal components of a medical device to the distal components of a medical device, according to an exemplary embodiment. As described above, wire 138 may include a proximal wire portion 138A and a distal wire portion 138B that releasably couple to one another. The proximal wire portion 138A and distal wire portion 138B may be connected by a first connector 300 and a second connector 400 of the connection assembly 600. The first connector 300 may include a hook 302 that receives a pin 402 of a second connector 400. In other embodiments, however, the first connector 300 may include a pin, and the second connector 400 may include a hook.

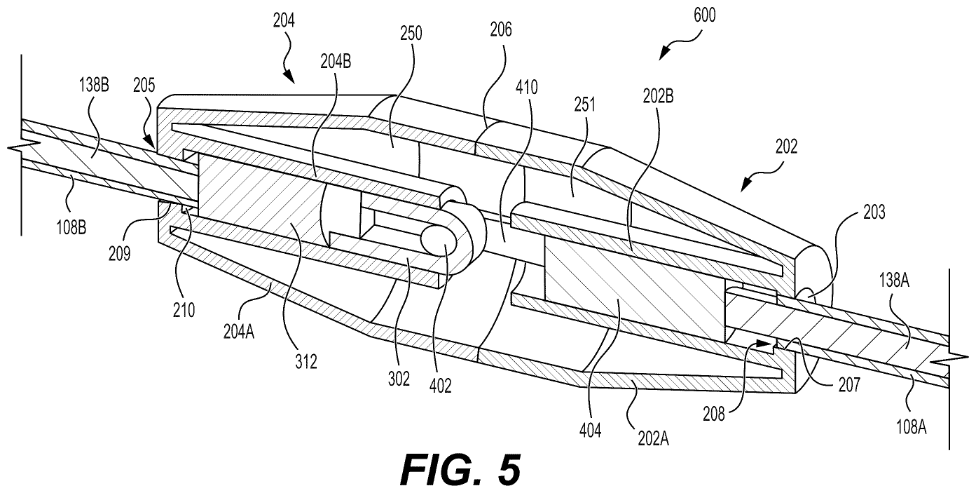

Connection assembly 600 may also include a proximal joint 202 and a distal joint 204. Proximal joint 202 may slide over proximal shaft portion 108A and wire portion 138A, and distal joint 204 slides over distal shaft portion 108B and wire portion 138B. Shaft portions 108A and 108B are not shown in FIG. 4, so that wire portions 138A and 138B are visible. After hook 302 and pin 402 are connected, proximal joint 202 may be slid distally over at least portions of second connector 400, and distal joint 204 may be slid proximally over at least portions of first connector 300, until proximal joint 202 and distal joint 204 meet and connect at a connection 206. In a final assembled form of connection assembly 600 (as shown in FIGS. 4 and 5), wire portion 138A may be secured to proximal joint 202, and the wire portion 138B may be secured to a distal joint 204. Connection assembly 600 may provide a releasable, indirect coupling of proximal shaft portion 108A to distal shaft portion 108B. As discussed in detail below, connection assembly 600, in the final assembled form, constitutes a pushing, pulling, and torque transferring mechanical coupling.

FIG. 5 shows a perspective, cross-sectional view of connection assembly 600 in a final, assembled, connected form. This disclosure will first describe first and second connectors 300,400, and their connection, followed by proximal and distal joints 202,204 and their connection (which is the chronological order of connections to produce an assembled connection assembly 600).

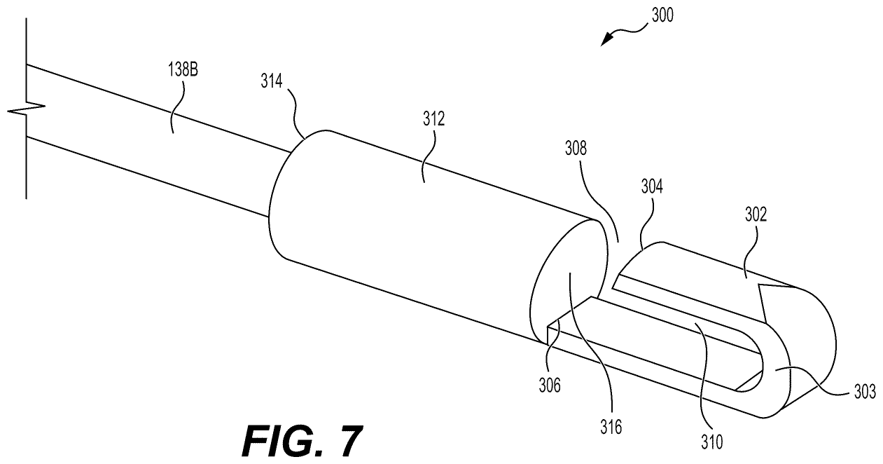

FIG. 7 shows a perspective view of first connector 300 and a proximal end of distal wire portion 138B. First connector 300 may be fixed to distal wire portion 138B via any suitable structure/method, including adhesive, weld, etc. Alternatively, first connector 300 may be integrally formed at the end of distal wire portion 138B.

First connector 300 may include a body 312 that may be cylindrical or any other suitable shape. Body 312 has a distal face 314 and a proximal face 316, each of which may be circular or otherwise corresponds to the shape of body 312. First connector 300 also includes hook 302, hook 302 may have an end 306 fixed to face 316 of body 312. Hook 302 may also include a curved portion 303 having a curvature to facilitate engagement with pin 402 (as will be described). Curved portion 303 may be positioned between end 306 and a free end 304 of hook 302. Free end 304 may be spaced from proximal face 316 to create a space 308 there between. Hook 302 may define a space 310 within hook 302. Space 310 may have a “U” shape between curved portion 303 and face 316. As described herein, spaces 308 and 310 may receive portions of second connector 400.

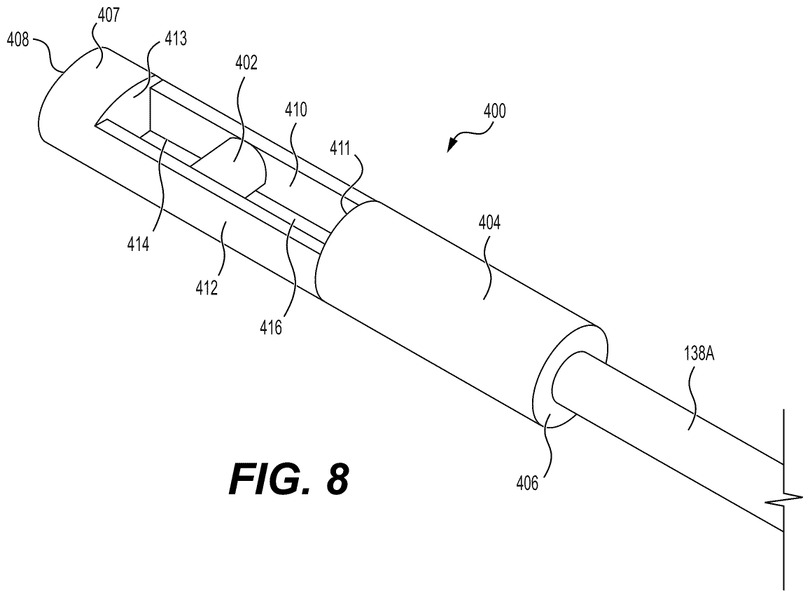

FIG. 8 shows a perspective view of second connector 400 and a distal end of proximal wire portion 138A. Second connector 400 may be fixed to proximal wire portion 138A via any suitable structure/method, including adhesive, weld, etc. Alternatively, second connector 400 may be integrally formed at the end of proximal wire portion 138A.

Second connector 400 may include a body 404 that may be cylindrical or any other suitable shape. Body 404 may have a distal face 411 and a proximal face 406, each of which may be circular or otherwise corresponds to the shape of body 404. Second connector 400 may also include two arms 410 and 412 extending longitudinally from body 404 to an end body 407. In the embodiment, arms 410 and 412 may be spaced apart. End body 407 may be cylindrical or any other suitable shape. End body 407 may have a distal face 408 and a proximal face 413, each of which may be circular or otherwise corresponds to the shape of end body 407. Arms 410, 412 may extend longitudinally from distal face 408 of end body 407. Pin 402 may be at a midpoint between distal face 411 of body 404 and proximal face 413 of end body 407. Pin 402 may extend between and connect to arms 410,412. Pin 402 may span a space defined by second connector 400. Span connector 400 may define two spaces 414,416. Space 414 is between pin 402 and end body 407, and space 416 is between pin 402 and body 404. Space 416 may receive curved portion 303 of hook 302. The portions of second connector 400 that bound space 416 form a “loop” that receives hook 302.

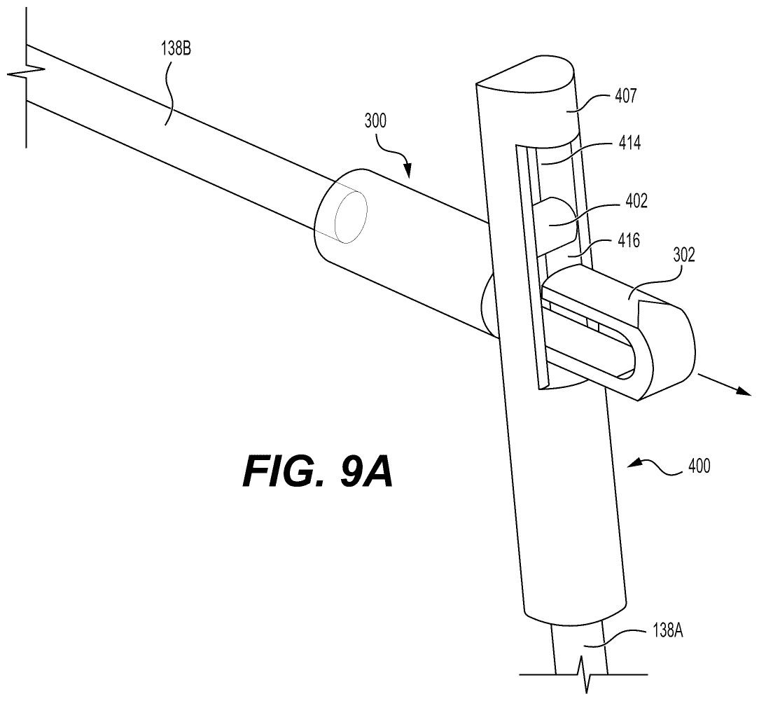

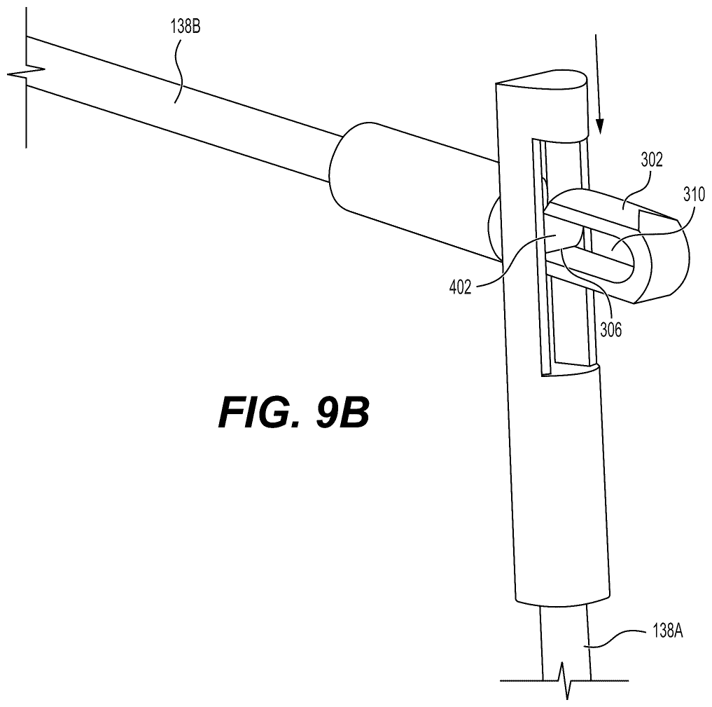

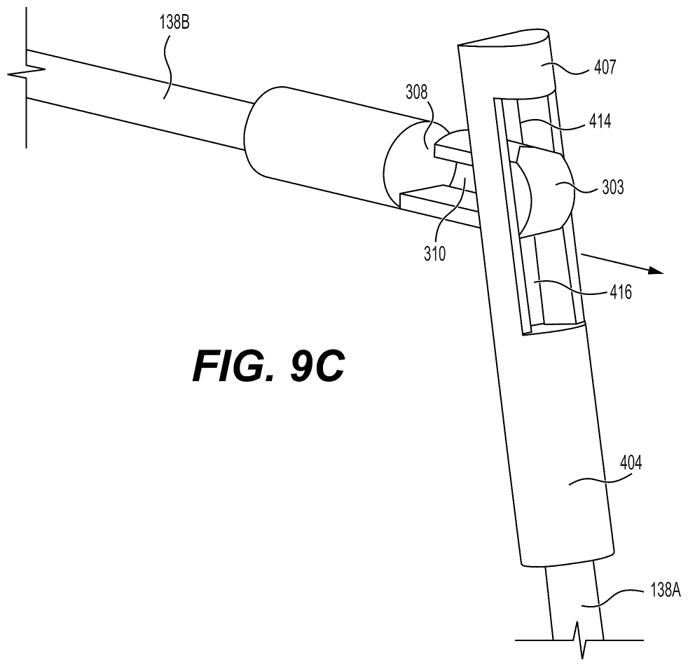

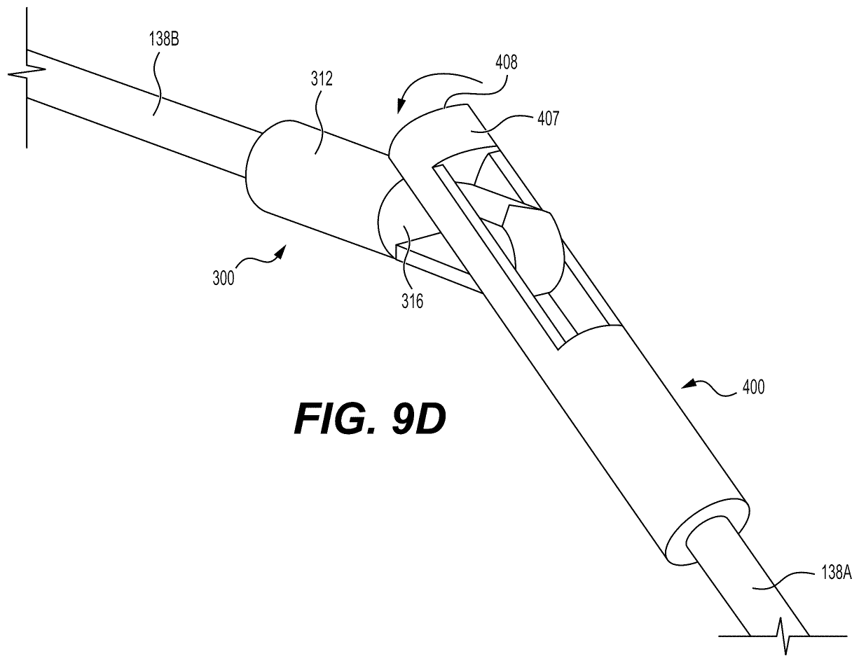

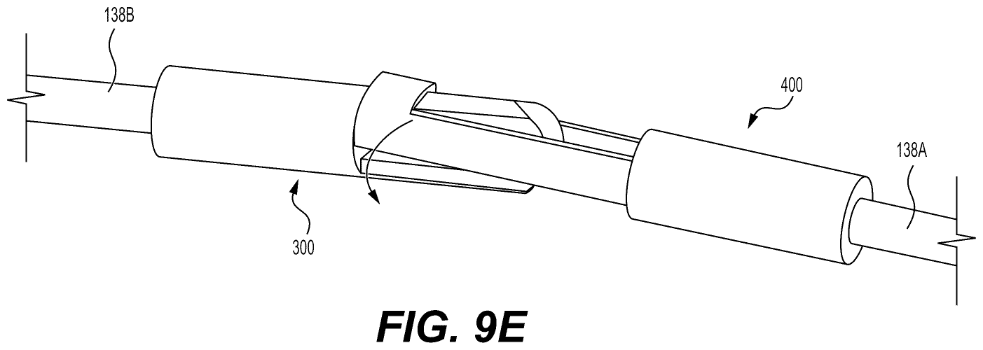

As shown in FIGS. 9A-9E, an exemplary method for a user to assemble first connector 300 and second connector 400 will be described. FIG. 9A shows a perspective view of first connector 300 and second connector 400. Second connector 400 may have an approximately vertical configuration, positioning proximal wire portion 138A beneath second connector 400, and first connector and distal wire portion 138B may be transverse to the configuration of second connector 400 and proximal wire portion 138A. First connector 300, particularly hook 302, may be introduced into space 416 until pin 402 is adjacent to space 308 of first connector 300.

As shown in FIG. 9B, pin 402 may be inserted through space 308 so that pin 402 enters hook 302. To do so, in the positions shown in FIG. 9B, second connector 400 may be transitioned directly downward relative to first connector 300. Connected end 306 may act as a stop for pin 402 during this step. Then, as shown in FIG. 90, second connector 400 may then be translated within space 310 until pin 402 contacts curved portion 303, creating a vacant space 308. As shown in FIGS. 9D and 9E, the user may rotate second connector 400 about pin 402 toward the first connector 300. In doing so, space 308 receives end body 407. Pin 402 may remain secured within space 310 and adjacent to curved portion 303.

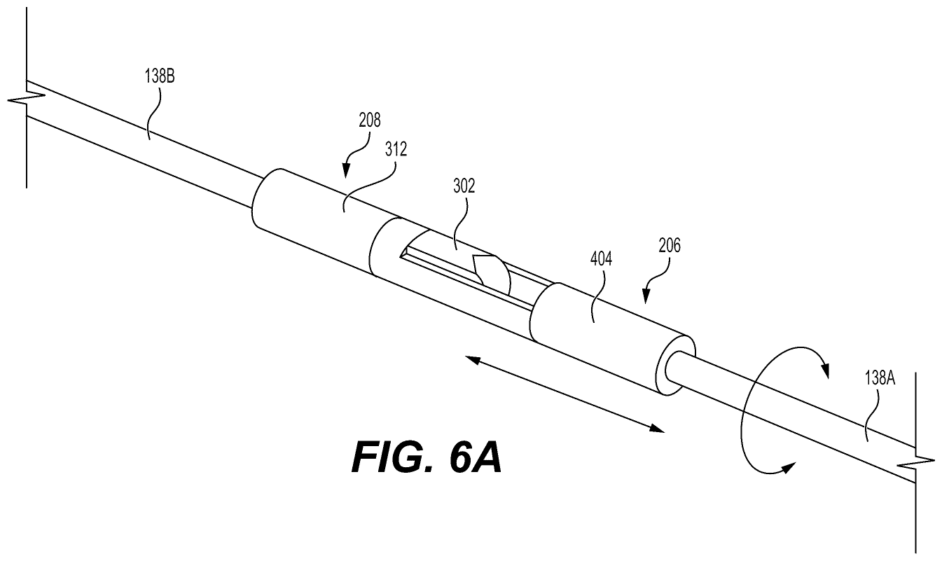

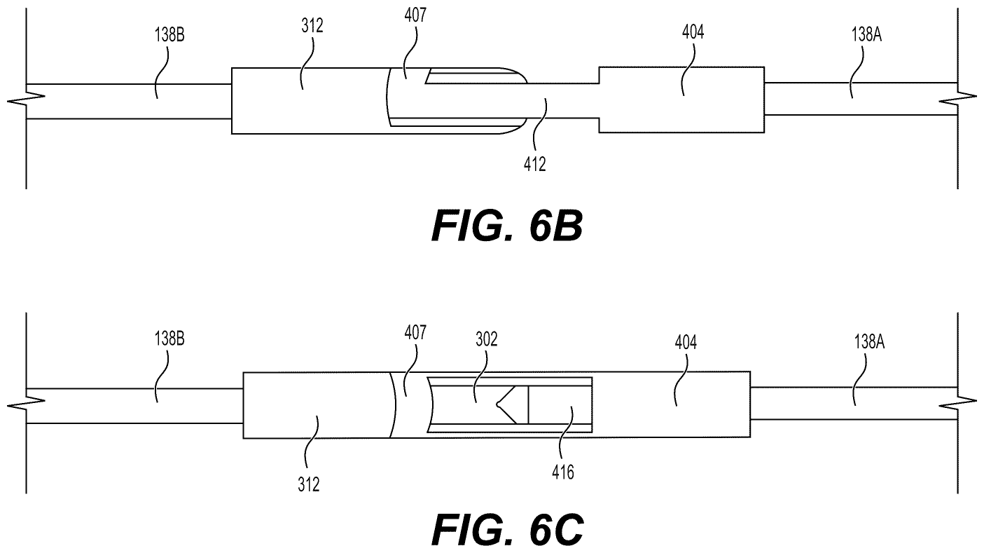

FIGS. 6A-6C show perspective, side, and top views respectively of the completed connection of first connector 300 to second connector 400 to releasably couple proximal wire portion 138A to distal wire portion 138B. As shown in these Figures, end body 407 may occupy space 308 and is adjacent to hook 302. A portion of space 416 may contain curved portion 303 and another portion of space 416 may be unoccupied.

As depicted by the arrows in FIG. 6A, the interlocked first and second connectors 300, 400 may receive pushing, pulling, and torque forces after coupling, to transfer such forces from proximal wire portion 138A to distal wire portion 138B. For example, knob 124 may rotate proximal wire portion 138A, and the connection between first connector 300 and second connector 400 may transfer the torque generated by knob 124 on proximal wire portion 138A to distal wire portion 138B. As yet another example, the connection between the first connector 300 and the second connector 400 may transfer a push and pull force transmission generated by spool 118 in a first direction onto the proximal wire portion 138A (e.g., in the distal direction) or a second direction (e.g., in a proximal direction) onto the distal wire portion 138B.

After connecting first connector 300 to second connector 400, the user may slide proximal joint 202 over proximal shaft portion 108A, proximal wire portion 138A, and at least part of second connector 400, and slides distal joint 204 over distal shaft portion 108B, distal wire portion 138B, and at least parts of first connector 300. The user may do this until proximal joint 202 and distal joint 204 meet and connect at a connection 206.

As shown in FIG. 5, distal joint 204 may include an annular outer wall 204A and an inner wall defining a cylindrical (tube shaped) sleeve 204B. Outer wall 204A may connect to sleeve 204B at their distal ends. Outer wall 204A may be integral with sleeve 204B. An annular space 250 may be between outer wall 204A and sleeve 204B. In other embodiments not shown, the space between outer wall 204A and sleeve 204B may be filled. A cylindrical space may be within sleeve 204B and, in a final assembled configuration of connection assembly 600, is filled with at least parts of each of first connector 300 and second connector 400.

At the distal end of distal joint 204, distal joint 204 may include an annular flange 209 extending radially inward from the inner wall defining sleeve 204B. Flange 209 defines a distal opening 205 of distal joint 204 and includes a proximally facing wall 210. Distal opening 205 may receive distal wire portion 138B and distal shaft portion 108B. Flange 209 may act as a seal around distal shaft portion 108B. Wall 210 may act as a stop surface so that distal joint 204 cannot slide over first connector 300. Wall 210 may contact face 314 of first connector 300, preventing disconnection of distal joint 204 from the distal components of the medical device.

Similar to distal joint 204, proximal joint 202 may include an annular outer wall 202A and an inner wall defining a cylindrical (tube shaped) sleeve 202B. Outer wall 202A may connect to sleeve 202B at their proximal ends. Outer wall 202A may be integral with sleeve 202B. An annular space 251 may be between outer wall 202A and sleeve 202B. In other embodiments not shown, the space may be between outer wall 202A and sleeve 202B may be filled. A cylindrical space may be within sleeve 202B and, in a final assembled configuration of connection assembly 600, is filled with at least parts of second connector 400.

At the proximal end of proximal joint 202, proximal joint 202 may include an annular flange 207 extending radially inward from the inner wall defining sleeve 202B. Flange 207 may define a proximal opening 203 of proximal joint 202 and may include a distally facing wall 208. Proximal opening 203 may receive proximal wire portion 138A and proximal shaft portion 108A. Flange 207 may act as a seal around proximal shaft portion 108A. Wall 208 may act as a stop surface so that proximal joint 202 cannot slide over second connector 400. Wall 208 may contact face 406 of second connector 400, preventing disconnection of proximal joint 202 from the proximal components of the medical device.

In the final assembled configuration of connection assembly shown in FIG. 5, Sleeve 204B may provide internal support to first connector 300 and second connector 400, preventing their disconnection. Specifically, sleeve 204B covers and contacts parts of each of first connector 300 and second connector 400 to prevent their pivoting/rotational movement relative to one another. Outer walls 202A and 204A may provide an enclosure for the inner connections, providing protection from the external environment.

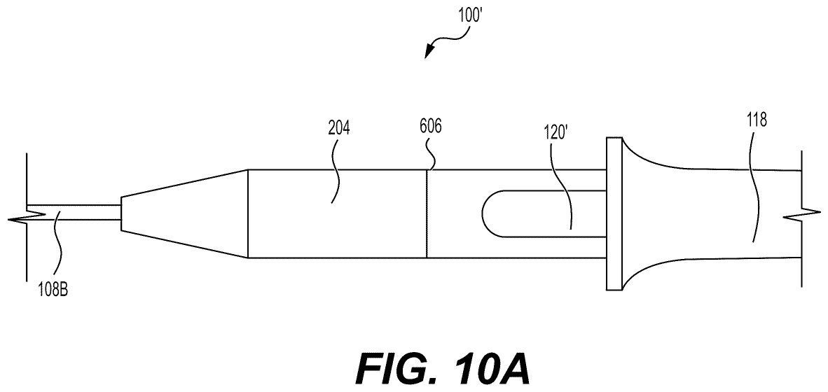

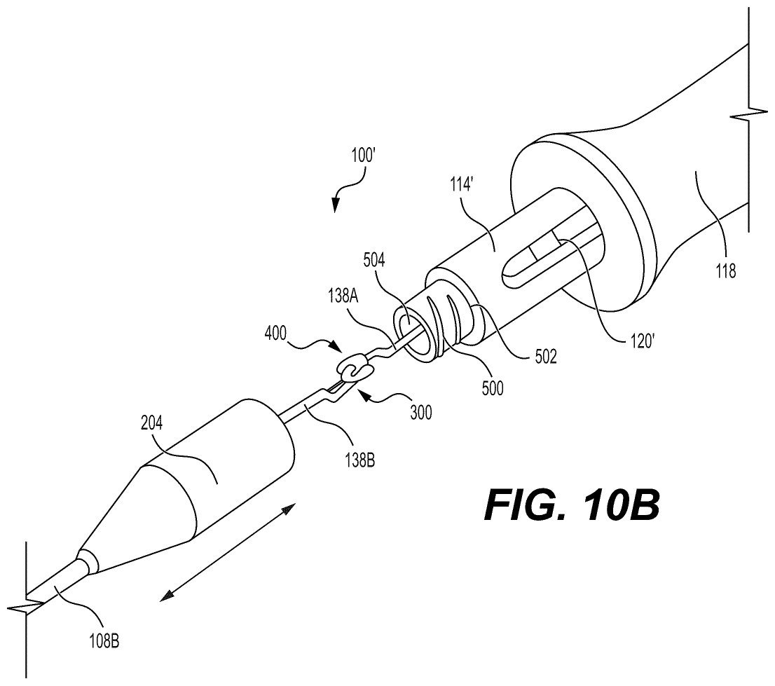

Connection 206 may be positioned between distal joint 204 and proximal joint 202 may be any suitable releasable mechanical connection. For example, connection 206 may be a snap fit connection having interlocking features holding distal joint 204 to proximal joint. The interlocking features can include or protrusions on one joint, while the other joint has corresponding slots or receptacles. When bringing the two joints together, the protrusions on one component may “fit” into the slots on the other component to assist in creating a secure connection 206. In another embodiment, a friction fit may connect joints 202, 204. In this embodiment, a snug fit may be created by proximal joint 202 and distal joint 204 with sufficient force that the friction between surfaces of the joints prevents separation. A threaded connection using male and female threads may be used in yet another embodiment, for example, the threaded connection discussed in more detail in FIG. 10A-10B.

Once the user has assembled connection assembly 600 as shown in FIGS. 4 and 5, the user may perform a procedure with medical device. After the procedure is completed, the user may disconnect the proximal components of medical device 100 from the distal components of medical device 100 by reversing the assembly steps described above in connection with FIGS. 4-9E. The user may then discard the distal components, which may have contacted internal parts of a patient. The proximal components can undergo a cleaning operation, if needed, and reused in a subsequent procedure with sterile, new distal components.

FIGS. 10A-10B illustrate another embodiment of a structure and method of connecting proximal components and distal components of a medical device 100′. FIG. 10A shows a partial side view of medical device 100′ illustrating the connection of the proximal components to the distal components, whereas FIG. 10B shows a partially connected state. Unless described otherwise, all structure and methods of assembly and operation of this embodiment are the same as the embodiment of FIGS. 1-9E. For example, all of the distal components of the embodiments are the same, including distal shaft portion 108B, distal wire portion 138B, first connector 300, and distal joint 204.

The main differences between the embodiment of FIGS. 10A-10B and the prior described embodiments are (1) medical device 100′ does not include proximal joint 202 or proximal shaft portion 108A, and (2) distal joint 204 connects directly to the handle of medical device 100′ at connector 500 of the handle to form a connection 606.

Body 114 of the handle includes connector 500 having external threads and an opening 504 for proximal wire portion 138A to extend from. Connector 500 includes a distally-facing stop surface 502 to contact a proximally-facing surface of distal joint 204. Distal joint 204 may include internal threads on its interior surface to mate with the external threads of connector 500.

An exemplary method for a user to assemble the proximal components to the distal components of medical device 100′ will now be described. A user may connect first connector to second connector as described above. The distal joint 202 then may be slid proximally over the connected first and second connectors and screwed onto connector 500 (using mating threads) until it reaches stop surface 502 and forms connection 606.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A proximal component of a medical device, the proximal component comprising:
a handle;
a coupling portion configured to releasably couple to a distal joint of a distal component of the medical device; and
a proximal wire extending distally from the handle, a distal end of the proximal wire including a connector configured to releasably connect to a distal wire of the distal component of the medical device.

2. The proximal component of the medical device of claim 1, wherein the connector includes a hook.

3. The proximal component of the medical device of claim 2, wherein the hook has a first end fixed to a face of a body of the connector, and the hook has a free end, wherein a curved portion of the hook is between the first end and the free end, and a space is defined between the free end and a distal face of the body.

4. The proximal component of the medical device of claim 1, wherein the connector includes a loop defined by a proximal body, a pin and two arms connecting the proximal body to the pin.

5. The proximal component of the medical device of claim 4, wherein the two arms are spaced apart and extend longitudinally from a distal face of the body, and the pin spans a space between the two arms.

6. The proximal component of the medical device of claim 5, wherein a first space is proximal to the pin, and the connector defines a second space distal to the pin and between the pin and a distal body of the connector.

7. The proximal component of the medical device of claim 6, wherein the pin is between a distal face of the proximal body and a proximal face of the distal body.

8. The proximal component of the medical device of claim 1, further comprising a proximal shaft portion over the proximal wire, wherein the proximal wire translates relative to the proximal shaft portion.

9. The proximal component of the medical device of claim 8, wherein the coupling portion includes a proximal joint that slides over the proximal shaft portion and the proximal wire.

10. The proximal component of the medical device of claim 9, wherein the proximal joint includes an outer wall and an inner wall defining a sleeve.

11. The proximal component of the medical device of claim 10, wherein the outer wall connects to the sleeve at a proximal end of the proximal joint, and a space is between the outer wall and the sleeve.

12. The proximal component of the medical device of claim 10, wherein the proximal joint includes a flange extending radially inward from the inner wall and defining a distally facing wall, wherein the flange defines a proximal opening of the proximal joint, wherein the proximal opening receives the proximal wire.

13. The proximal component of the medical device of claim 10, wherein the sleeve surrounds and contacts the connector.

14. The proximal component of the medical device of claim 9, wherein the proximal joint rotates relative to the proximal shaft portion.

15. The proximal component of the medical device of claim 1, wherein the handle includes the coupling portion at a distal end of the handle.

16. A distal component of a medical device, the distal component comprising
an end effector;
a distal wire extending proximally from the end effector, a proximal end of the distal wire including a connector configured to releasably connect to a proximal wire of a proximal component of the medical device;
a distal shaft portion over the distal wire, wherein the distal wire translates relative to the distal shaft portion; and
a distal joint configured to releasably couple to a proximal joint of the proximal component of the medical device, wherein the distal joint translates over the distal shaft portion.

17. The distal component of the medical device of claim 16, wherein the connector includes one of a hook and a loop.

18. The distal component of the medical device of claim 16, wherein the distal joint includes an outer wall and an inner wall defining a sleeve, wherein the outer wall connects to the sleeve at a distal end of the distal joint, and a space is between the outer wall and the sleeve, wherein the distal joint defines a distal opening that receives the distal wire.

19. A medical device, comprising:
a proximal handle;
a distal end effector;
a proximal wire extending distally from the handle, a distal end of the proximal wire including a first connector;
a distal wire extending proximally from the distal end effector, a proximal end of the distal wire including a second connector to releasably connect to the first connector;
a proximal coupling portion over the proximal wire; and
a distal joint that translates over the distal wire and is configured to releasably couple to the proximal coupling.

20. The medical device of claim 19, wherein the first connector includes one of a pin and a hook, and the second connector includes the other of the pin and the hook.
